(19) United States
(12) Patent Application Publication
(10) Pub. No.: US 2016/0345893 A1
(43) Pub. Date: Dec. 1, 2016

(54) WEARABLE DEVICE AND SYSTEM FOR STOPPING AIRWAY DISORDERS INCLUDING SUCH A WEARABLE DEVICE

(71) Applicant: Nitetronic Holding Limited, Grand Cayman (KY)

(72) Inventors: Hubertus Edler von Janecek, Shanghai (CN); Johannes Herrnsdorf, Herdecke (DE)

(21) Appl. No.: 15/166,693

(22) Filed: May 27, 2016

(30) Foreign Application Priority Data
May 28, 2015 (EP) 15169605

(51) Int. Cl. A61B 5/00; A61F 5/56; A61B 5/1455; A61B 5/087; A61B 7/00; A61B 5/01

(52) U.S. Cl. CPC A61B 5/4818; A61B 5/682; A61B 5/6822; A61B 5/6819; A61B 5/6831; A61B 7/003; A61B 5/01; A61B 5/14552; A61B 5/087; A61B 5/0002; A61F 5/56; A61B 5/4836; A61B 2562/0204; A61B 2562/0219; A61B 2562/0247

(57) ABSTRACT

A wearable device for detecting body characteristics and for use in an airways disorder alleviating system includes a device housing, a means for attaching the device housing to a body part of a sleeping individual, one or more sensors configured to detect body characteristics, and a communication unit configured to communicate at least one of information of body characteristics and a respiratory disorder event information indicating that a respiratory disorder event is ongoing or upcoming to an external device.

### TECHNICAL FIELD

The present invention relates to a system, particularly a head support, for alleviating a respiratory disorder event on humans. In addition to that, the invention relates to a method for sensing and detecting body characteristics of a sleeping individual. For clarification, the term individual refers to a human person, the term body refers to a body of a human.

### RELATED ART

A head support for stopping snoring of a sleeping individual is known by document U.S. Ser. No. 14/358,625, wherein the head support has a head resting surface for the head of a sleeping individual to rest on. The head support has an active layer with an arrangement of neighboring deforming elements for controlling the height of the head support section by section. To detect a position of the head resting on the head resting surface a sensing layer is provided which is arranged between the head resting surface and the active layer.

During sleep of an individual, pathologic effects such as snoring and/or sleep apnea can reduce oxygen absorption which may be dangerous to health of the sleeping individual. It has been found that moving head or body of the sleeping individual during snoring or during an ongoing sleep apnea event may immediately resolve the snoring or sleep apnea event, respectively, and urges the individual to continue normal breathing.

From prior art, various approaches are known to detect irregularities in respiration of a sleeping individual due to sleep apnea and trying to actively move the body and/or the head of the sleeping individual.

In document WO 2014/114438 A1, a sleeping pillow for alleviating a sleep apnea is disclosed. The sleeping pillow has an adjusting means system which can be actuated for changing the position of a head resting on the sleeping pillow by way of a control device and which has sensor means for detecting the position of the head. The control device is configured so that in addition sensor means signals from further sensor means for detecting respiratory activities of a person can be detected. In order to detect a sleep apnea of a person using the sleeping pillow, the further sensor means comprise sensors for detecting a breathing frequency or breathing movements of the chest of the person or sensors for detecting the nose dynamic pressure of a person. The adjusting system means systems can be actuated with respect to the sensor means signals of the further sensor means to alleviate or to avoid a sleep apnea event.

From document DE 10 2009 039 915 A1, an apparatus for the detection and signaling of the sleep-relating breathing dysfunction of an individual is known. Therein, it is described that a sensor is fixated at the head of an individual so that mechanical inertia at the head of the individual can be measured and analyzed to obtain head alignment information, breathing information about a breathing behavior and snoring information about the snoring behavior of the individual.

Document CN 103 222 909 A discloses a pillow with air chambers. Between the surfaces, a sensor is provided which is connected to a processor. The processor is configured to collect a sleep respiratory rate of a user. The sensor is configured as a sound sensor for acquiring the number of breaths, breath frequency rate and other sleep information from the user.

Document CN 102 743 246 A discloses an inflatable pillow with air chambers for changing the position of a head of an individual resting on the pillow. When a sleep apnea occurs, the individual's head is moved by locally actuating the air chambers of the pillow. Detection of the sleep apnea may be performed by means of a wrist assembly for detecting the patient's pulse and blood oxygen concentration, means for detecting a sleeping position or by means of a belly assembly for detecting a swing and abdomen abdominal breathing gas flow component.

Document CN 1557270 A discloses a pillow with inflatable air chambers which are controlled depending on an input of a respiratory sensor signal.

Document KR 100711701 B1 discloses a pillow for preventing a sleep-related breathing disorder. The pillow has a number of chambers in which a pressure can be regulated. By detecting the characteristics of pressure in each of the chambers, it can be estimated whether a breathing disorder is caused by comparing the pressure of each chamber with control data indicating an optimal pressure pattern data. The pressure of the air chambers is regulated, if a breathing disorder is detected.

It is therefore an object of the present invention to provide a robust and reliable detection of an airway disorder during sleep of an individual and to move a head of a sleeping individual depending on the detection of an airway disorder event.

While a detection of snoring resulting from a respiratory disorder can be easily detected using microphones, detecting an upcoming or ongoing airways disorder event, e.g. a sleep apnea, may not be remotely sensed with ease. It is therefore a further object of the invention to provide means to measure further body characteristics which allow a monitoring of other body functions and to provide this information to an airways disorder alleviating device.

### SUMMARY OF THE INVENTION

The objects listed above can be resolved by the wearable device for detecting body characteristics, the airways disorder alleviating system and the head support described herein.

According to a first embodiment a wearable device for detecting body characteristics and as well for use in an airways disorder alleviating system comprises:
a device housing;
a means for attaching the device housing to a body part of a sleeping individual;
at least one sensor configured to detect body characteristics and
a communication unit configured to communicate at least information of body characteristics and respiratory disorder event information, for indicating that a respiratory disorder event is ongoing or upcoming to an external device.

The above wearable device is configured to detect body characteristics of a sleeping individual whose head rests on a head support. The wearable device can conveniently be worn at least on one part of a human body. It is configured to be located at positions which are found being comfortable for a sleeping individual and since it abuts to the skin of at least one body part of the individual it is configured for continuously monitoring body characteristics for detecting an upcoming and/or ongoing airways disorder event, such as a snoring or a sleep apnea event.

The body characteristics can be analyzed in the wearable device or sensor signals of the body characteristics can be transmitted to a control unit of the head support. Therein the body characteristics are processed to detect an upcoming and/or ongoing respiratory disorder event. The wearable device can be worn closely to the head or neck of the sleeping human individual and is not limited to only detecting snoring noise, but also further body characteristics, e.g. airways airflow, airflow humidity, airflow alcohol level, blood oxygen saturation, the heart rate and the orientation of a head resting on a head support (side or supine alignment in a lying position of the individual). The information about the body characteristics can be transmitted to a control unit which analyzes the body characteristics and which constitutes whether and in which manner a movement of the head of the sleeping individual shall be initiated in order to suppress or stop the respiratory disorder event.

According another embodiment of the invention, information can be transmitted to a monitoring device for monitoring body characteristics during sleep of the individual.

According another embodiment of the invention, means for attachment are configured to attach the device housing to at least the upper side of the head of a sleeping individual, the forehead of a sleeping individual, the neck of a sleeping individual, to an area located between the nose and the mouth, and the chest of a sleeping individual, wherein means for attachment comprise at least one of a belt, a strap and a stripe.

The information of the body characteristics comprise at least one of:
a snoring noise,
vibration or vibrations of a device housing,
an orientation of a device housing,
a body temperature of a sleeping individual,
a blood oxygen level,
a heart rate
airflow humidity,
airflow alcohol level, and
a pressure sensor and its reading referring to a respiratory activity of a sleeping individual.

The at least one sensor comprises at least of one or more of:
a microphone configured to detect a snoring noise,
an inertia sensor configured to detect vibrations and/or an orientation of the device housing,
a temperature sensor configured to detect the body temperature of a sleeping individual,
a blood sensor configured to detect a blood oxygen level and/or a heart rate,
a pressure sensor configured to detect a respiratory activity of a sleeping individual;
a humidity sensor configured to detect an airflow humidity;
an alcohol sensor configured to detect an airflow alcohol level.

The inertia sensor in combination with any one of the pressure sensor, the blood sensor and the microphone can be used in combination with a head support interacting with the head or body of a sleeping individual. The detection of the head orientation in combination with body characteristics in a single device makes the device easy to use, especially in combination with the external device configured to move the head or any part of the body of a sleeping individual in order to stop or to suppress a respiratory disorder event.

According another embodiment of the invention, a pressure sensor is connected via a nasal air pipe to the nose of the sleeping individual.

According to another embodiment an inertia sensor detects an orientation of the head or body of a sleeping individual wherein the communication unit transmits information about this orientation to an external device. The inertia sensor provides the orientation of the head or the body as additional information for evaluating other body characteristics.

According to another embodiment, a head support comprises:
a resting surface for the head of a sleeping individual;
an active layer configured to separately deformation of the resting surface in secluded sections;
a control unit configured to receive at least one of information of body characteristics and to determine a respiratory disorder event information indicating that a respiratory disorder event is ongoing or upcoming based on the at least one of information of body characteristics and to actuate the active layer.

A control unit may be configured to receive information about the orientation of the head or the body of a sleeping individual, of at least one of information of body characteristics and to actuate the active layer.

The control unit may be configured to receive information about the breathing activity of the sleeping individual, of at least one of information of body characteristics and to actuate the active layer.

According to another embodiment of the invention, an airways disorder alleviating system comprises a wearable device and a head support.

### BRIEF DESCRIPTION OF THE DRAWINGS

The embodiments of the invention are described in detail in conjunction with the drawings, in which:

FIG. 1 schematically shows an airways disorder alleviating system;

FIG. 2 schematically shows possible locations for attachment of the wearable device;

FIG. 3 schematically shows a further possible location for attachment of the wearable device including a nasal air pipe;

FIG. 4 schematically shows a further possible location for attachment of a wearable device in proximity of the nose of a sleeping individual;

FIG. 5 schematically shows a block diagram characterizing a wearable device; and

FIGS. 6a and 6b illustrate a use of the wearable device in order to detect the orientation of the head of the sleeping individual.

### DESCRIPTION OF EMBODIMENTS

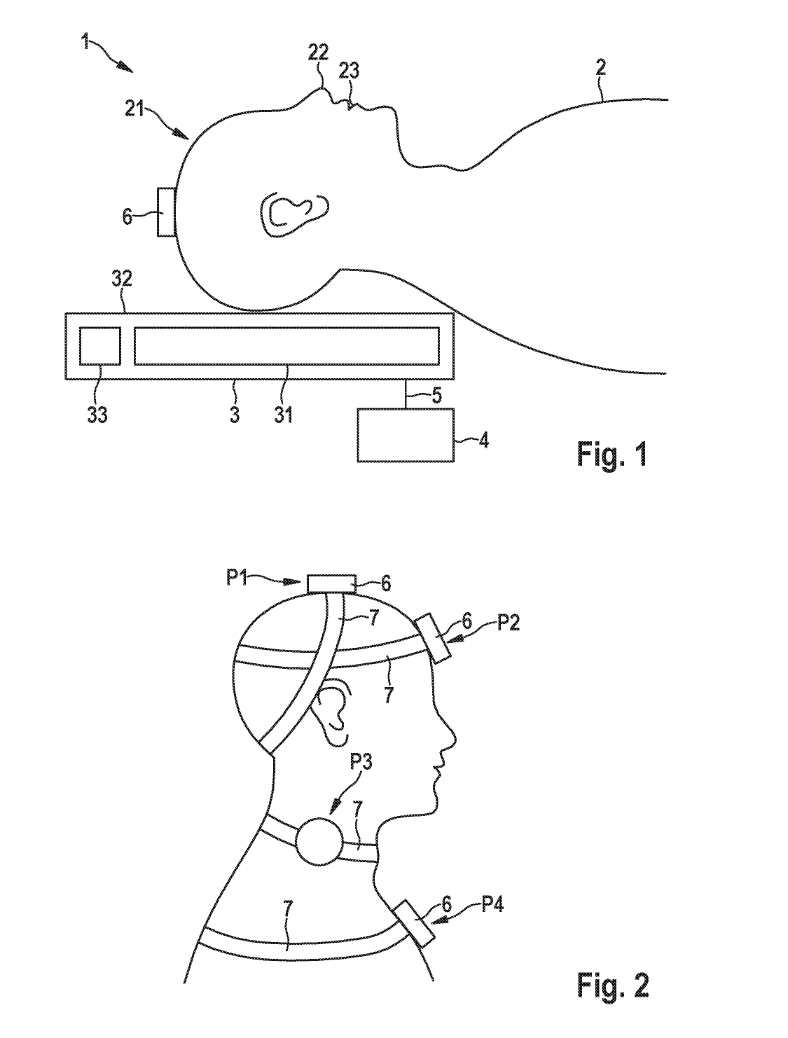

FIG. 1 shows an airways disorder alleviating system 1 which is configured to stop an ongoing and/or upcoming airways disorder event of a sleeping individual 2. An airways disorder event is any kind of respiratory irregularity, such as snoring or a sleep apnea event.

The system 1 includes a head support 3, e.g. a pillow, on which a head 21 of the individual 2 rests while an individual is sleeping. The head support 3 may include a variety of components which include an active layer 31 which allows for separate deformation of at least one resting surface 32 of the head support 3 in secluded sections so that the height of the resting surface 32 can be individually set. The active layer 31 may include deformation elements (not shown) which are closely neighbored or distanced from one another in order to adjust the height of the resting surface 32 of the head support 3 in at least one of the sections the head of a sleeping individual 2 is resting in order to change the orientation and/or position of the head 21 in a predetermined manner.

To identify the deformation elements to be actuated to move the head of the sleeping individual 2, the position of the head 21 can be detected by a position detection means 33 in the head support 3. Alternatively, position detection means can be located externally to the head support 3.

A control unit 4 may be located externally outside the head support 3 and connects therewith via an interconnection 5. In an alternative embodiment, the control unit 4 can be arranged internally the head support 3.

Onto the body of a sleeping individual 2, a wearable device 6 may be attached which is configured to detect at least one body characteristics and to transmit the body characteristics continuously to the control unit 4. The transmission of information referring to body characteristics is either wireless or cable-bound, however, a wireless connection between a control unit 4 and the wearable device 6 is of preferential use for allowing the head of a sleeping individual to freely move on the head support. Communication protocols for transmission to the control unit 4 include WiFi, Bluetooth or similar technologies. An optical data transmission via LiFi using invisible light is preferable as electromagnetic radiation for data transmission can be reduced.

FIG. 2 shows alternatives for arranging the wearable device 6 onto the body of an individual 2. Possible locations for attachment of the wearable device 6 are on the upper side of the head (position P1), on the forehead (position P2), on the neck (position P3) and on the chest (position P4). The wearable device 6 can be attached to the body by means of belts, straps or stripes 7 so that a displacement of the wearable device 6 is prevented. The materials of the belts, straps or stripes 7 comprise plastics, rubber and/or metal, and may be elastic. The attachment is such that one surface of the wearable device 6 abuts the skin of a sleeping individual 2, facilitating detection of body characteristics.

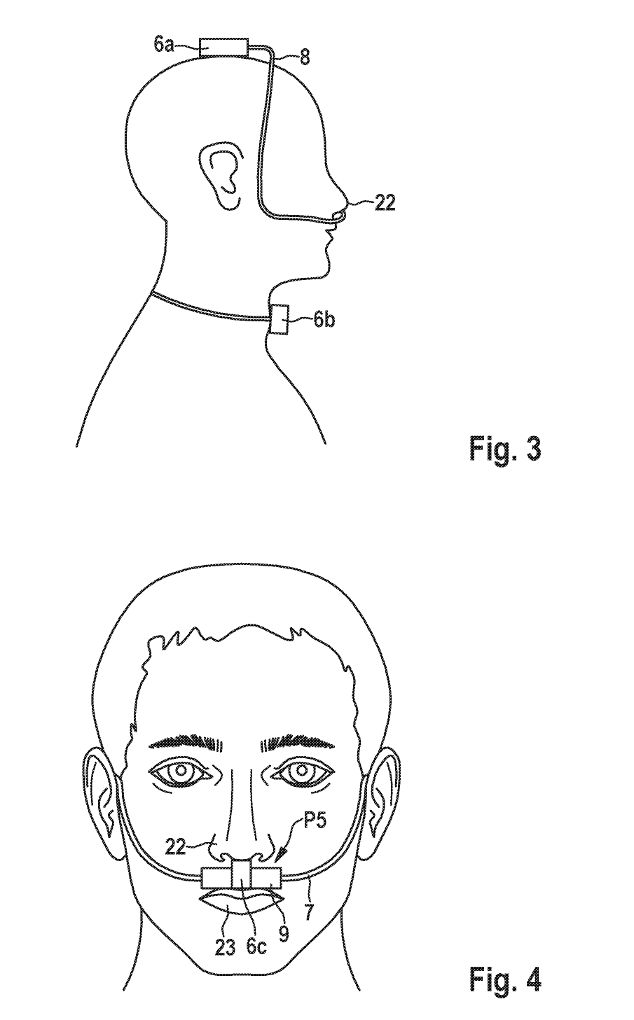

Another embodiment of the invention is configured to combine multiple wearable devices 6 comprising different sensors which are arranged on different positions P1, P2, P3, P4, P5. FIG. 3 shows the arrangement of a first wearable device 6a at an upper portion of the head or at the side of the head, which is connected by a nasal air pipe 8 of the nose 22 of a sleeping individual 2 such that a pressure change in the air pipe 8 resulting from the breathing of the individual can be detected by a corresponding pressure sensor in the wearable device 6.

Another embodiment of the invention is shown in FIG. 7 wherein another wearable device 6d is worn in proximity of a nose 22 of a sleeping individual. In order to hold the wearable device 6d belts, straps or stripes 7 extend between the ears and are held in position by a loop around the head or by an attachment onto the ears. The wearable device 6d may include the pressure sensor 40 among none, or at least one or more other sensor.

As shown in FIG. 8, the pressure sensor 40 as used in the embodiments of FIGS. 2, 3, 4, and 7 may be connected with the nose airways of a sleeping individual via the nasal air pipe 8. The nasal air pipe 8 may be disposably connected to a first end of a tube 41 extending from the wearable device 6, 6a, 6b, 6c, 6d, wherein a second end of the tube 41 has an opening arranged close to a pressure sensor unit 44 fixated on a substrate 42. The tube 41 is arranged parallel to the sensing surface of the pressure sensor unit 44 and attached thereon by means of a glob-top 43 (e.g. epoxy resin) which is a piece of glue which allows a pressure change in the interior of the tube 41 to be propagated to and detected by the sensing surface of the pressure sensor unit 44. This arrangement allows for use of disposable nasal air pipes 8 which finds more acceptance due to hygienic reasons. The attachment of the tube 41 on the pressure sensor unit 44 by means of a glob-top allows a flexible connection between the tube 41 and the pressure sensor unit 44 which may better accept movements which may occur between the pressure sensor 40 and the nasal air pipe 8 due to movement of a sleeping individual 2.

A second wearable device 6b comprising a vibration sensor can be alternatively or additionally arranged at the neck of a sleeping individual 2 so that vibration can be detected which can be used in order to determine that a snoring detected by microphones arranged remotely from the head of the sleeping individual originates from the individual on the head support 3.

A third wearable device 6c or a part, i.e. a sensor, thereof, can, as shown in FIG. 4, alternatively or additionally be arranged in proximity of the nose 22 of a sleeping individual 2, particularly between the nose 22 and the mouth 23 of a sleeping individual 2. This has the advantage that airflow through both the nose 22 and the mouth 23 can be detected with the same sensor unit, such as an airflow sensor.

The third wearable device 6c can include a microphone to acoustically measure the airflow, a pressure sensor to measure pressure variations caused by breathing or a temperature sensor to measure nose airflow by temperature change.

The third wearable device 6c can include a carrier plate 9 made of soft plastic, rubber, textile or foam to be located closely onto the skin at a location (position P5) between the nose 22 and the mouth 23 so that the breathing air can flow along the plate 9.

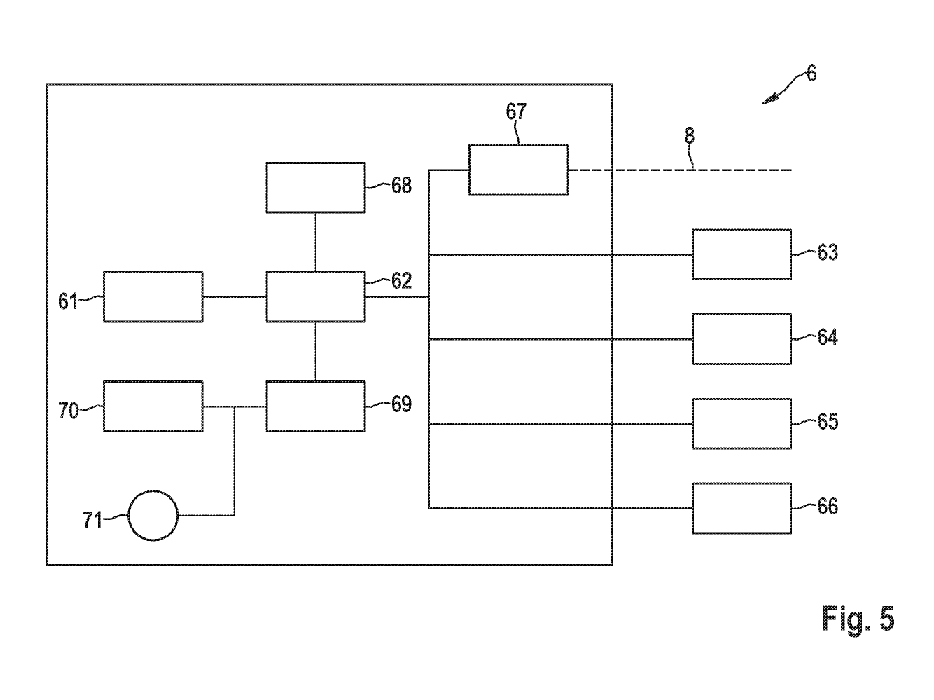

FIG. 5 shows a block diagram illustrating the functionality of the wearable device 6. The wearable device 6 comprises a communication unit 61 for wirelessly transmitting information of body characteristics to the control unit 4.

A controller 62 is provided which is connected to at least one or a combination of sensors 63-67 for obtaining signals therefrom. For illustration the wearable device 6 is shown with a combination of sensors 63-67. Any combination of sensors 63-67 can be configured in the wearable device 6.

The controller 62 is configured to be connected with a memory 68 to store program code, to be executed by the controller 62, and to store information about body characteristics and with a power supply 69 to supply power to operate the controller 62 and the sensors 63-67 for acquiring body characteristics. The power supply 69 can be charged by a power plug 70 and/or an antenna for wireless charging.

The sensors 63-67 may include:
a microphone 63 for detecting a snoring noise made by a sleeping individual 2,
an inertia sensor 64 to detect vibrations and/or an orientation of the wearable device and so of the body, particularly the head, neck or chest of a sleeping individual 2,
a temperature sensor 65 to detect the body temperature of a sleeping individual,
a blood sensor 66 to detect the blood oxygen level and/or a heart rate;
a pressure sensor 67, which may be arranged directly or via the air pipe 8 with an airflow through the nose 22 of a sleeping individual 2, so that the respiratory activity of a sleeping individual 2 can be detected.

In one embodiment of the invention, the wearable device 6 is connected to the head or neck of a sleeping individual 2 and may include a combination of sensors such as, a microphone 63, an inertia sensor 64 and a pressure sensor 67. This arrangement has the advantage that a snoring noise can be detected and by the pressure sensor 67 the breathing activity can be monitored so that an airways disorder event can be determined by the controller 62 or by the control unit 4. An inertia sensor 64 determines the orientation of the head 21 such that the actuation of the active layer 31 in the head support 3 occurs, depending on the actual orientation of the head 2. If the inertia sensor 64 has sufficient sensitivity vibrations caused by snoring are detected, thus the microphone might be omitted.

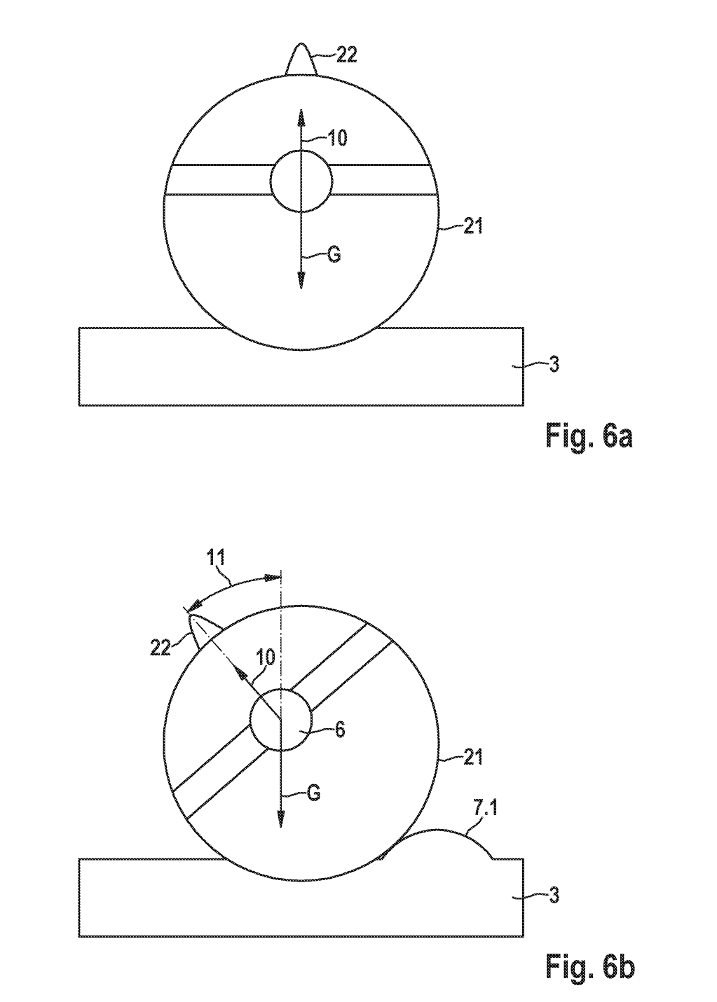

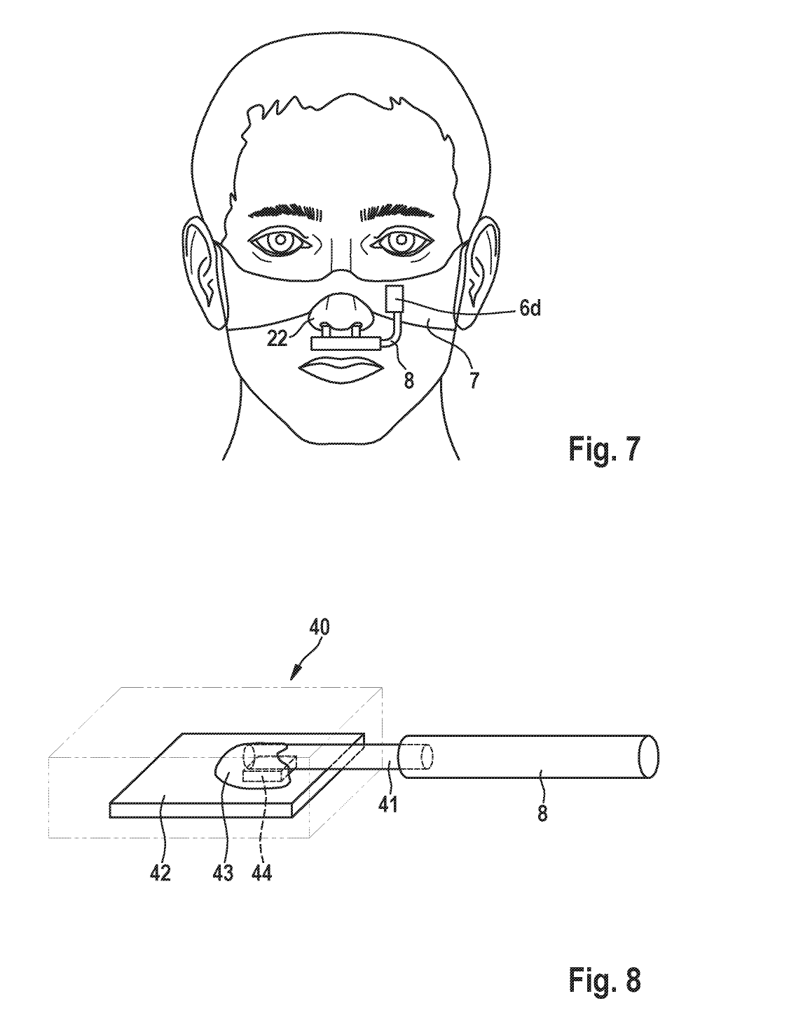

By means of the inertia sensor 64, a turning of the head 21 due to an actuation of the head support 3 can be monitored so that a feedback can be obtained by which angle the head 21 of a sleeping individual 2 resting on the head support 3 is/has been tilted. This is illustrated in FIGS. 6a and 6b for two different orientations of the head of a sleeping individual 2. The inertia sensor 64 measures its own spatial orientation due to the direction it is exposed to gravity G. The detection of the orientation of the head 21 of a sleeping individual 2 determines the direction into which the head 21 of a sleeping individual 2 is to be turned and the angle by which the head 21 is to be turned so that the movement of the head 21 can be fully controlled, whenever a respiratory disorder event has been detected.

The sensor data obtained by the inertia sensor 64 is used to verify that a snoring detected by the sensor data of the microphone 63 origins from the individual wearing the wearable device 6.

The controller 62 performs an analysis of the data provided by one or more of the sensors 63-67 and uses the communication unit 61 to transmit upcoming or ongoing respiratory disorder event information to the control unit 4. The control unit 4 activates the active layer 31 in the head support 3 depending on information received.

The communication unit 61 submits the information about the body characteristics as detected by one or more of the at least one sensor 63-67 via a suitable transmission protocol, such as WiFi, Bluetooth or similar to the control unit 4 of the head support 3. Hence, the communication unit 61 in an alternative embodiment connects to a portable device, such as a mobile device, via a standard transmission protocol, such as WiFi, Bluetooth or the like, which monitors the information of body characteristics so that characteristics about information of the body characteristics during the sleep of the individual are obtained via an app operated on the mobile device.

1. A wearable device for detecting body characteristics and for use in an airways disorder alleviating system, comprising:
a device housing;
a means for attaching the device housing to a body part of a sleeping individual;
one or more sensors configured to detect body characteristics; and
a communication unit configured to transmit at least one of information of body characteristics and a respiratory disorder event information indicating that a respiratory disorder event is ongoing or upcoming to an external device.

2. The wearable device according to claim 1, wherein the means for attaching are configured to allow the device housing to be attached to at least one of an upper side of the head of the sleeping individual, a position at a forehead of the sleeping individual, a position at a neck of the sleeping individual, a location between the nose and the mouth, a position at a chest of the sleeping individual, and a position aside the nose, wherein the means for attaching particularly comprise at least one of a belt, a strap and a stripe.

3. The wearable device according to claim 1, wherein the information of body characteristics comprises at least one of:
a snoring noise,
vibrations and/or an orientation of the device housing,
a body temperature of the sleeping individual,
a blood oxygen level and/or a heart rate,
a pressure sensor related to a respiratory activity of the sleeping individual.

4. The wearable device according to claim 1, wherein the one or more sensors comprise one or more of:
a microphone configured to detect a snoring noise,
an inertia sensor configured to detect vibrations and/or an orientation of the device housing,
a temperature sensor configured to detect the body temperature of the sleeping individual,
a blood sensor configured to detect a blood oxygen level and/or a heart rate,
a pressure sensor configured to detect a respiratory activity of the sleeping individual.

5. The wearable device according to claim 1, wherein the one or more sensors include a pressure sensor that is connected via a nasal air pipe to the nose of the sleeping individual.

6. The wearable device according to claim 1, wherein a longitudinal tube is arranged in parallel to a sensing surface of a pressure sensor unit of the pressure sensor so that an opening at a first end of the tube is disposed at the sensing surface, wherein a second end of the tube is disposably attachable to the nasal air pipe.

7. The wearable device according to claim 1, wherein the one or more sensors includes an inertia sensor configured to detect an orientation of the head or body of the sleeping individual wherein the communication unit is configured to transmit an information about the orientation to an external device.

8. The wearable device according to claim 7, wherein an airflow sensor is provided to detect a respiratory activity of the sleeping individual.

9. A header support comprising:
a resting surface for a head of a sleeping individual to rest on;
an active layer configured to allow a separate deformation of the resting surface in sections;
a control unit configured to receive at least one of information of body characteristics and to determine a respiratory disorder event information indicating that a respiratory disorder event is ongoing or upcoming based on the at least one of information of body characteristics and to actuate the active layer depending on the at least one of information of body characteristics.

10. The header support according to claim 9, wherein the control unit configured to receive an information about the orientation of the head or the body of the sleeping individual as the at least one of information of body characteristics and to actuate the active layer depending on the information about the orientation of the head or the body.

11. The header support according to claim 9, wherein the control unit is configured to receive an information about a breathing activity of the sleeping individual as the at least one of information of body characteristics and to actuate the active layer depending on the information about the breathing activity.

12. (canceled)

13. An airway disorder alleviating system comprising:
a wearable device comprising:
a device housing;
a means for attaching the device housing to a body part of a sleeping individual;
one or more sensors configured to detect body characteristics; and
a communication unit configured to transmit at least one of information of body characteristics; and
a head support comprising:
a resting surface for a head of a sleeping individual to rest on;
an active layer configured to allow a separate deformation of the resting surface in sections; and
a control unit configured to receive the at least one of information of body characteristics from the communication unit and to determine a respiratory disorder event information indicating that a respiratory disorder event is ongoing or upcoming based on the at least one of information of body characteristics and to actuate the active layer depending on the at least one of information of body characteristics.
